SEQUENCE LISTING (9 sequences)

SEQ ID NO: 1 - DNA, 678 bp, Bugula Neritina
gctcggacccccagcagcgcttgttgttacaggagtcatggaatgctctggaggatgccgggtatggacaagaagatatgagtaacaaagtattggtatgtttgtgggtgcggagacgggtgattatcagggtttagcaagtggcagtaatattacatccaatcataatgctatgcttgctgcacggctggcgtattttctcaatctcagtggtccggtgatgtccattgatactacctgttcctccgctcttgttgctttacatcaagcctgtgtgaacttgcgtcagcacgaatgtgatactaccatcgtagctgttaatttatgctttgcattggaaacctatcagacattgagtcacgcaggtatgttatccccggatggcaaatgctttacctttgataagcgggccaatggtatggtccctggagaagcggtcgtcgcggtggtacttaaacccctctctcaggccattgcagacggtgatcccgtctatgccagcattcgtggtagtggccttaactacgatggaaaaactaatggcattaccgctcccagtggcacagcgcaaacagcgctgttgaaacaggtgtatgaacaggcgcaagtggatgttgaacacattgactatattgtagcgcatggcaccggaacccaactgggcgacccaatcgaact

SEQ ID NO: 2 - protein, 226 aa, Bugula Neritina
SDPQQRLLLQQSWNALEDAGYGQEDMSNKSIGMFVGAETGDYQGLASGSNITSNHNAMLAARLAYFLNLSGPVMSIDTTCSSALVALHQACVNLRQHECDTAIVAGVNLCFALETYQTLSHAGMLSPDGKCFTFDKRANGMVPGEAVVAVVLKPLSQAIADGDPVYASIRGSGLNYDGKTNGITAPSGTAQTALLKQVYEQAQVDVEHIDYIVAHGTGTQLGDPIE

SEQ ID NO: 3 - DNA, 705 bp, Bugula Neritina
atggacccgcagcaacggttatttttagagaatgcgtggagttgtatggaggatgcggggattaaccctaagatgttatcccgtagtcgatgtggggtatttgttggggtgcggtgcgaatgattacagcgctctaatgaacagtagccactcaacgagtctcgaattaatgaaggaattaggcaacaactcttccatttatctgcacgaatctcctactttttaaatttaaagggcccttgtcttgcgattgataaccgcatgctcttcttcattagtggccattgccgagtcgtgtaatagtctggtgttgggtactagtgacttggcgttggcaggtggagtgttgctgatgccaggtvvatcctcatatataggcttgagtcatggagaaatgttatcagtagatggtcgctgctttaccttcgatcaacgtgccaacggttttgtacctggagagggtgtaggggttgtcttgttaaaacgcatgtcggatgcggtgcgtgatggtgatcccattcgtgcagtgatacggggctggggtgtgaatcaggatggtagaagtaatggtattacggcgccgagttcaaaggcgcaaagtgctctggagcaagaggtttatcaacgttttaatattgatccatcgagcattaccttagtcgaagcacacggaaccggcaccaaactgggcgatcccatcgagct

SEQ ID NO: 4 - protein, 234 aa, Bugula Neritina
MNPQQRLFLENAWSCIENAGINPKMLSRSRCGVFVGCGANDYSALMNSSHSTSLELMKELGNNSSILSARISYFLNLKGPCLAIDTACSSSLVAIAESCNSLVLGTSDLALAGGVLLMPGPSLHIGLSHGEMLSVDGRCFTFDQRANGFVPGEGVGVVLLKRMSDAVRDGDPIRAVIRGWGVNQDGRSNGITAPSSKAQSALEQEVYQRFNIDPSSITLVEAHGTGTKLGDPIE

SEQ ID NO: 5 - protein, 6 aa, Artificial
  Primer
MDPQQR

SEQ ID NO: 6 - protein, 6 aa, Artificial
  Primer
LDPQQR

SEQ ID NO: 7 - protein, 6 aa, Artificial
  Primer
SDPQQR

SEQ ID NO: 8 - protein, 7 aa, Artificial
  Primer
LGDPIEI

SEQ ID NO: 9 - protein, 7 aa, Artificial
  Primer
LGDPIEL

(19) United States
(12) Patent Application Publication
(10) Pub. No.: US 2007/0026488 A1
(43) Pub. Date: Feb. 1, 2007

(54) Biosynthesis of bryostatins by polyketide synthases (PKS)

(76) Inventors: Nancy M. Targett, Lewes, DE (US); Nicole B. Lopanik, Ann Arbor, MI (US)

Correspondence Address: Basil S. Krikelis;Citizens Bank Center, 919 N. Market Street, Suite 1800, P.O. Box 111, Wilmington, DE, 19899, US

(21) Appl. No.: 11/190,406

(22) Filed: Jul. 27, 2005

(51) Int. Cl. C12P 21/06; C07H 21/04; C07K 14/195; C12N 1/21

(52) U.S. Cl. 435/69.1; 435/320.1; 435/252.3; 435/325; 530/350; 536/23.5

(57) ABSTRACT

The present invention relates to the discovery, isolation and sequencing of novel polyketide synthase genes. These newly identified genes are involved in the biosynthesis of bryostatins. The genes are found to be expressed in all life stages of Bugula neritina, are associated with the bacterial symbiont Endobugula sertula and are believed to be part of a large polyketide gene cluster that is responsible for the production of bryostatins.

### RELATED FEDERALLY SPONSORED RESEARCH

The work described in this application was sponsored by the Sea Grant under Contract Number NA96RG0029 and NA56RG0147.

### FIELD OF THE INVENTION

The present invention relates to newly discovered Polyketide Synthase genes responsible for biosynthesis of bryostatins.

### SEQUENCE LISTING

This application includes the submission of a single disc containing Sequence Listings in computer readable form. The disc is labeled as follows: “Docket No. 99689-00015; Applicant: Nancy M. Targett, et al.; Title: Biosynthesis of Bryostatins by Polyketide Synthases (PKS); Format: MS WORD (Notepad); SEQUENCE LISTING; Date Created: Jul. 27, 2005; Size 7 kb”. All of the information contained in this disc is hereby incorporated by reference in its entirety.

### BACKGROUND OF THE INVENTION

For many communities in the marine environment, factors that affect larval survival can directly influence the adult population structure of an organism. High mortality at the vulnerable larval and post-settlement stages has led to the evolution of defenses that can increase the chance of survival. Thus, any advantage a specific larva has over others may impart greater success to the future adult. Many of the factors that affect larval success can also impact post-settlement or juvenile success. Mortality can be high once larvae have settled and metamorphosed and can therefore affect the population structure.

Because clonal sessile marine invertebrates are functionally like plants, they may be affected by similar evolutionary pressures including consumption by mobile predators, competition for space, and the inability to move to other habitats in unfavorable environmental conditions. Like plants, marine invertebrates have long been the source of novel compounds that reduce the impact of some of these pressures. Secondary metabolites may act as signaling molecules for settlement and food sources, antimicrobial agents, antifouling agents, anti-competitor agents, and feeding deterrents. As such, much of the focus on chemical defenses has been on identifying active compounds.

For many years it was assumed that eggs and embryos could not be chemically defended because the presence of toxic secondary metabolites was contrary to normal metamorphosis and development for the vulnerable early stages. However, defensive secondary compounds such as unpalatable metabolites, have been found marine invertebrate eggs and larvae of sponges, soft corals, hydroids, polychaetes, echinoderms, bryozoans, and ascidians. The presence of such chemical defenses in post-settlement larvae and juveniles has not been studied extensively.

Recently, variation in chemical defenses among distinct life stages of certain marine invertebrates have been documented; in particular, the concentrations of deterrent metabolites in the larval stage may be very different than those in the adult stage. Of particular interest, the larvae of the marine bryozoan Bugula neritina are unpalatable to co-occurring vertebrate and invertebrate predators, while the adult does not have the same deterrent chemistry. Understanding this ontogenetic difference in the chemical defense of B. neritina can result in the better understanding of the controls on the production of deterrent metabolites.

Bugula neritina is an erect, branching bryozoan found in temperate habitats throughout the world, and is often considered a nuisance, fouling organism. B. neritina is a cage-captor bryozoan, i.e., an individual zooid twists the tops of its tentacles together and uses the cage to capture and contain prey. Bugula neritina (Bryozoa) often co-occurs with the bacterial endosymbiont Candidatus Endobugula sertula. Many populations of B. neritina produce a biologically active class of polyketide-derived secondary metabolites called bryostatins, which have shown significant activity in cancer therapy. Their ecological role is less well-defined; however, because adult B. neritina extracts are palatable to sympatric predators but their larval extracts are not, it has been suggested that B. neritina larvae are chemically defended by deterrent bryostatins.

The bryostatins are polyketides, a class of acetate-derived compounds isolated from different populations of B. neritina. Interest in the secondary chemistry of B. neritina was piqued when, as mentioned above, researchers found that extracts of the bryozoan inhibited the growth of cancer cell lines. The first bryostatin compound discovered, bryostatin 1, is presently undergoing Phase II anticancer clinical trials (Varterasian et al. 2000, 2001, Bedikian 2001, Pfister et al. 2002). To date 20 different compounds classified as bryostatins have been isolated from populations of B. neritina throughout the world. In each case, however, the bryostatins can only be obtained from B. neritina in very low concentrations.

It has been established that B. neritina larvae contain significant concentrations of multiple bryostatins. It also appears that B. neritina sequesters the bryostatins on the outside of the growing embryo and larvae. It has further been determined that the bacterial symbiont, Candidatus Endobugula sertula (that is present in both the larval and adult stages) produces the deterrent bryostatins present in the larvae. This is the first account of a microbial symbiont biosynthesizing a chemical that protects its marine invertebrate host from predators.

Currently, many of the bryostatins used in medical research are extracted from adult B. neritina grown in aquaculture operations. The finding that B. neritina larvae have such high concentrations of bryostatins has implications for the pharmaceutical industry because B. neritina larvae could be a continuous and renewable source of bryostatins, thus greatly reducing the cost and effort associated with extraction of these compounds. Additionally, the finding that the bryostatins are produced by an endosymbiont may also benefit the pharmaceutical industry because the symbiont could potentially be cultured or the compounds could be produced in a heterologous host and could result in greater yields of these compounds.

A putative bryostatin PKS from California-based B. neritina/E. sertula populations has been sequenced and characterized (D. Sherman, pers. comm.), but because E. sertula has not been cultured to date, there are no studies to definitively demonstrate that the characterized PKS is responsible for the production of the bryostatins.

The symbiosis of E. sertula/B. neritina is unique in that there are no examples in the literature in which there are various concentrations of symbiont-produced secondary metabolites in different ontogenetic stages of a marine invertebrate. The difference in concentrations of bryostatins in adult and larval tissue leads to the question of whether only symbionts in the larval tissue are producing the metabolites, or if the symbionts in both adult and larval tissue are synthesizing the bryostatins, which are somehow being concentrated in the larvae.

It is now well dcoumented that yields of bryostatins from Bugula neritina are extremely low. Existing methods for the recovery of bryostatins from Bugula neritina require the destructive harvesting and processing of the organism. Some methods have been proposed and tested for the harvesting of the Bugula neritina for bryostatin acquisition, such as collecting wild Bugula neritina growing in natural coastal environments. However, as reported by the National Cancer Institute to acquire sufficient quantities of bryostatin 1 to conduct Phase I clinical trials, the National Cancer Institute had to collect and process approximately 13,000 kilograms of Bugula neritina in order to obtain approximately 18 grams of bryostatin.

Concerns about environmental impacts resulting from over-collecting and the possible decimation of local populations of Bugula neritina have led to the desire to develop alternative methods for growing and harvesting the organism. One response to this need was the development of man-made aquacultural environments to grow Bugula neritina for harvesting and processing for bryostatins. While these man-made aquacultural environments may appear to be promising and offer some hope that large amounts of Bugula neritina organisms may be grown to meet the eventual biomedical and other research and commercial demands for bryostatins, they are expensive, and are susceptible to failure at several points in the process. A technique known as “clip-harvesting” wherein Bugula neritina is grown on plates suspended in ocean waters has also been proposed as an alternative harvesting method, whereby, the entire Bugula neritina organism is not destroyed. This technique allows the Bugula neritina to re-grow after a portion is clipped from each colony thus potentially cutting back the costs associated with re-seeding with new Bugula neritina each year. However, the costs of clip harvesting, while possibly lower than the destructive harvesting, are still high. In addition, the purification process itself is laborious and expensive.

Processing of the bryostatins is also a laborious process. The levels of bryostatins in Bugula neritina are typically in the range of 10⁻⁶ to 10⁻⁸ percent wet mass of the animal. During processing, the bryostatins are extracted from the Bugula neritina along with numerous other compounds produced by the animal. The bryostatins must then be purified from the complex mixture of chemicals in this extract. The purification of bryostatins from the Bugula neritina extract requires a great number of man-hours, the use of large amounts of chemicals, and expensive processing equipment, such as preparative-scale chromatography systems. Solvents typically used in the processing steps create health and safety hazards, pose environmental concerns, and increase the costs of processing. In addition, large amounts of solvent treated Bugula neritina organisms must be disposed of following processing. The overall costs of processing Bugula neritina to obtain bryostatins are very high, thereby making the acquisition of commercial quantities of bryostatins from Bugula neritina uneconomical while limiting the amount of bryostatin produced annually.

A cost-effective and commercially scalable synthesis of bryostatins has not yet been developed, which has further hindered the biomedical development and commercialization of the bryostatins. Although numerous research groups are currently working on developing methods for the efficient, large-scale and economical synthesis of bryostains, the structural complexity of the bryostatins has prevented the development of economically viable synthesis and thus the commercial development of the bryostatins.

Finding the biosynthetic gene would have several advantages: it can be transferred to a heterologous host which can produce under controlled conditions either the compound or the enzyme for in vitro synthesis of the metabolite; and the gene could be manipulated to engineer metabolites with desirable functional groups.

Therefore it would be a benefit in the art to identify a biosynthetic gene likely responsible for the production of the bryostatin compounds and quantify the levels of expression of a putative biosynthetic gene in the ontogenetic stages concurrent with the levels of defensive metabolites in the tissues. Some of this knowledge could be applied to obtaining a bioactive compound on a larger scale, whether it is collection of certain life stages of the invertebrate, or expression of the biosynthetic gene in a heterologous host. Knowing patterns of production of these bioactive compounds in marine invertebrates may also result in a more directed search for new metabolites.

### SUMMARY OF THE INVENTION

The present invention is directed to an isolated nucleic acid molecule consisting of a nucleotide sequence selected from the group consisting of:

(a) a nucleotide sequence that encodes an amino acid sequence shown in SEQ ID NO:2;

(b) a nucleotide sequence that hybridizes under stringent conditions to a nucleic acid molecule shown in SEQ ID NO:1; and

(c) a nucleotide sequence that encodes a fragment of an amino acid sequence shown in SEQ ID NO:2, wherein said fragment comprises at least 10 contiguous amino acids.

The invention is further directed to An isolated nucleic acid molecule consisting of a nucleotide sequence selected from the group consisting of:

(a) a nucleotide sequence that encodes an amino acid sequence shown in SEQ ID NO:4;

(b) a nucleotide sequence that hybridizes under stringent conditions to a nucleic acid molecule shown in SEQ ID NO:3; and

(c) a nucleotide sequence that encodes a fragment of an amino acid sequence shown in SEQ ID NO:4, wherein said fragment comprises at least 10 contiguous amino acids.

### BRIEF DESCRIPTION OF THE DRAWINGS

FIG. 1 provides a comparative analysis of the amino acid sequences of the PKS genes BKSc1 and BKSc3.

FIG. 2 shows a minimum evolution tree of amino acid sequences of PKS genes from B. neritina/E. sertula and other related organisms.

FIG. 3(A) shows the expression of PKS genes BKSc1 and BKSc3 (lanes 1-4 and lanes 5-8, respectively).

FIG. 3(B) shows that the PKS gene BKSc1 is present only in B. neritina larvae with the symbiont E. sertula (lanes 1-2; lanes 3-5 show that BKSc1 is not present in B. neritina larvae without E. sertula).

FIG. 4 provides a log of the ratios of B. neritina COI, E. sertula 16S and BKSc1 gene expression in B. neritina larval, adult reproductive and adult non-reproductive tissue.

### DETAILED DESCRIPTION OF THE INVENTION

The present invention relates to the discovery, isolation and sequencing of novel polyketide synthase genes which are responsible for the biosynthesis of bryostatin. The genes are found to be expressed in all life stages of B. neritna, and are believed to be part of a large polyketide gene cluster that is responsible for the production of bryostatin. It is the inventors' position that these genes will be extremely valuable in providing a relatively simple means for obtaining large-scale production of bryostatin 1, a promising anti-cancer drug.

In a preferred embodiment, the present invention is directed to novel ketosynthase (KS) genes that were isolated from B. neritina/E. sertula DNA. Two of the novel KS genes that we identified are referred to throughout this document as BKSc1 (SEQ ID NO. 1) and BKSc3 (SEQ ID NO. 3). The KS genes of the invention were found to encode PKS polypeptides. Two polypeptides of the invention are encoded by BKSc1 and BKSc3 and are provided herein as SEQ ID NOs 2 and 4 respectively. The PKS polypeptides of the invention, in their protein form, are known to catalyze at least one step in the synthesis of Bryostatin.

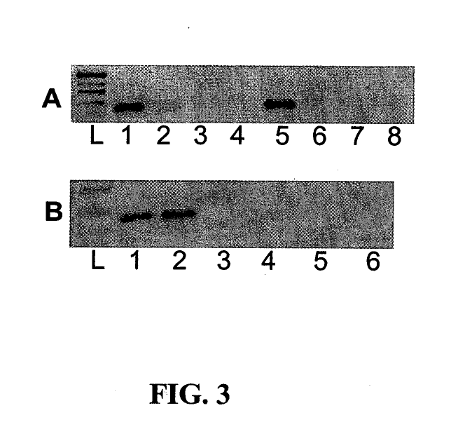

The novel KS genes of the invention were found to be expressed in larval and adult B. neritina/E. sertula RNA (see FIG. 3(A)). Further, the elimination of the E. sertula (also referred to throughout the application as the “symbiont”) from B. neritina adults and larvae resulted in the elimination of the KS genes of the invention (see FIG. 3(B)). Such a definitive determination that these KS genes are responsible for producing the bryostatins required cultivation of the symbiont, and such has not previously been accomplished in the art.

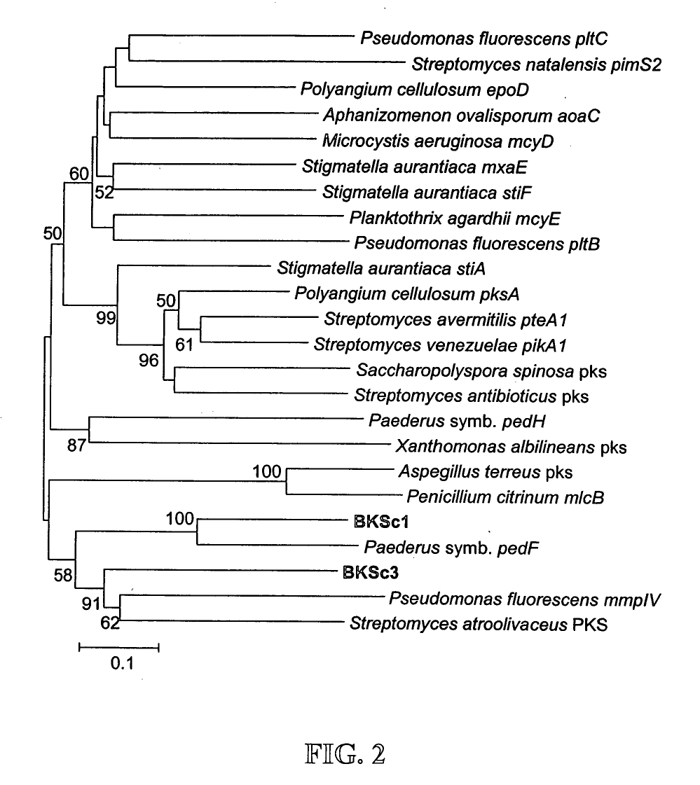

We have determined that the two KS genes of the present invention are homologous to other known KS genes isolated from bacteria, including γ-Proteobacteria (e.g., Paederus symbiont and Pseudomonas fluorescens) (see Example 1, Table 1). FIG. 2 depicts the homologies of the two novel KS genes of the present invention as compared to other related organisms. For instance, BKSc1 most closely resembles one of the KS genes from the Paederus spp. symbiont, which is an analogous host/symbiont system in the terrestrial environment. The symbiont is a Pseudomonad, a γ-Proteobacteria, like E. sertula, and produces a compound, pederin, which defends the larvae against predatory spiders. The B. neritina/E. sertula KS genes of the invention also, however, match KS genes from bacteria that are not closely related according to 16S rDNA sequences, i.e., Microcystis aeruginosa (cyanobacteria) and Streptomyces spp. (Actinobacteria). Additionally, the KS genes from two eukaryotes (Aspergillus terreus and Penicillium citrinium) are fairly homologous to the B. neritina/E. sertula KS genes of the invention. This is not surprising because prior research focusing on the evolution of aromatic polyketides in Streptomyces has shown that the phylogenetic relationships of the KS DNA did not follow the phylogeny based on 16S rDNA. These data suggest that the evolution of PKS genes is independent of the evolution of 16S rDNA, and that environmental pressures may play a role in the production of secondary metabolites.

In the B. neritina symbiont, E. sertula, it is known that there are 4 base pair differences in 16S SSU rDNA in populations from North Carolina and California. Populations collected from deep locations in California had one genotype, while populations collected from shallow locations less than a mile away had another genotype. Individuals with the “deep” genotype produced bryostatins 1-3, while populations with the “shallow” genotype produced bryostatins 4-11. These genotype/chemotype combinations corresponded with B. neritina cytochrome oxidase I (COI) genetic sequence variations, with an 8.1% difference between the sequences, indicating that the populations are in fact sibling species. We isolated our KS genes from the North Carolina B. neritina/E. sertula and found that these unique genes resided on the tree with other KS genes from a diverse group of organisms. The depth of diversity of the KS genes among the B. neritina sibling species from different geographical regions is not known. Because the evolution of PKS genes may be more influenced by biotic and abiotic environmental pressures, the PKS genes in the sibling species may be more divergent than the B. neritina COI gene and E. sertula 16S rDNA gene.

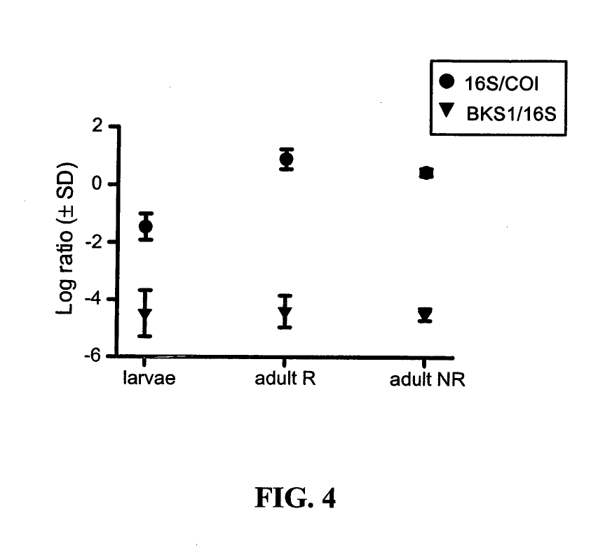

Because the bryostatins play such an important role in the defense of B. neritina larvae, and the concentrations in adult tissues are very low, where the compounds are produced in the tissue becomes an issue. Levels of expression of the KS genes of the invention in larval, adult reproductive and adult non-reproductive tissue in B. neritina/E. sertula were measured using quantitative real-time PCR. Numbers of E. sertula cells (as measured by expression of 16S rRNA) were higher in both reproductive and non-reproductive adult tissues than in larvae (FIG. 4). However, expression of the novel KS genes of the present invention was constant among the tissue types.

While it has been documented that bryostatins appear to be concentrated on the surface of the larvae, where predators can detect them without harming the larvae, the mechanism responsible for “storing” the bryostatins on the outside of the larvae is not known. The occurrence of secondary metabolites on the surface of some marine algae has been demonstrated to reduce fouling on the blades of the algae. The B. neritina/E. sertula association provides a unique example in the marine environment of how the symbiont can benefit the host by the production of deterrent secondary metabolites.

In the identification process, the KS genes of the invention were identified as small portions of the putative bryostatin biosynthetic. These genes appear to be associated with E. sertula, as the genes were not amplified from DNA from aposymbiotic B. neritina larvae (FIG. 3(B)). The expression of these genes was quantified in different ontogenetic stages to determine if PKS expression level was correlated with bryostatin concentrations. There was no clear difference in PKS gene expression in larval tissue vs. adult and juvenile tissues, indicating that either the bryostatins are produced constitutively throughout the ontogenetic stages of B. neritina. Because these genes are involved in bryostatin biosynthesis and the bryostatins are biosynthesized at low levels in both larval and adult B. neritina, there must be a mechanism for the transport of the compounds to the larvae. Because in the adults the symbiont E. sertula is found in the funicular cords, which provide nutrients to the developing embryo in the brood chamber, it seems likely that the bryostatins are transported to the larvae via the funicular system. Whether they are compartmentalized in vacuoles similar to phenolic compounds in brown algal vesicles (physodes) is not known, but this mechanism would reduce the exposure of B. neritina cells to the toxic bryostatins.

Throughout the remainder of this document, any KS gene of the invention shall be generally referred to as a nucleic acid molecule or a polynucleotide of the invention, while the polypeptide or protein encoded by the KS genes shall be generally referred to as a polypeptide or protein of the invention.

As used herein, the term “nucleic acid molecule” is used in a general sense to refer at least one of ribonucleic acid (RNA), ribonucleotide, deoxyribonucleic acid (DNA), deoxyribonucleotide, nucleic acid analog, synthetic nucleotide analogs, nucleic acid conjugates, for example peptide nucleic acids or locked nucleic acids, nucleic acid derivatives, polymeric forms thereof, and includes either single- or double-stranded forms. Also, unless expressly limited, the term “nucleic acid” includes known analogues of natural nucleotides that have similar binding properties as the reference nucleic acid. In addition, a particular nucleotide or nucleic acid sequence includes conservative variations based on the nucleotides adenine (“A”), guanine (“G”), cytosine (“C”), thymine (“T”), uracil (“U”), and inosine (“I”).

As used herein, the term “Polynucleotide” refers to a polymeric form of nucleotides of at least ten bases in length, either ribonucleotides or deoxyribonucleotides or a modified form of either type of nucleotide. The term includes single and double stranded forms of DNA or RNA.

As used herein, the term “Isolated polynucleotide” refers to a polynucleotide of genomic, cDNA, PCR or synthetic origin, or some combination thereof, which by virtue of its origin, the isolated polynucleotide (1) is not associated with the cell in which the isolated polynucleotide is found in nature, or (2) is operably linked to a polynucleotide that it is not linked to in nature. The isolated polynucleotide can optionally be linked to promoters, enhancers, or other regulatory sequences.

As used herein, the term “Polypeptide” is used herein as a genetic term to refer to native protein, fragments, or analogs of a polypeptide sequence.

As used herein, the term “Isolated protein” referes to a protein of cDNA, recombinant RNA, or synthetic origin, or some combination thereof, which by virtue of its origin the isolated protein (1) is not associated with proteins normally found within nature, or (2) is isolated from the cell in which it normally occurs, or (3) is isolated free of other proteins from the same cellular source, for example, free of cellular proteins, or (4) is expressed by a cell from a different species, or (5) does not occur in nature.

As used herein, the term “Percentage of sequence identity” is calculated by comparing two optimally aligned sequences over the window of comparison, determining the number of positions at which the identical nucleic acid base occurs in both sequences to yield the number of matched positions, dividing the number of matched positions by the total number of positions in the window of comparison (for example, the window size), and multiplying the result by 100 to yield the percentage of sequence identity.

Nucleic acid molecules of the present invention can be made using methods known in the art and described herein (see, Examples). For instance, nucleic acid molecules of the present invention can be identified and isolated using PCR methodologies, including RT-PCR, and sequenced using established methods such that their homologies can be determined. The ability of one nucleic acid molecule to hybridize with another can be determined through experimentation under a variety of stringencies, or an be estimated based on their length and G:C contents. Alterations of identified sequences can be made using routine methods, such as multagenesis, RT-PCR or other PCR methods as are well known in the art.

A nucleic acid molecule of the present invention can include at least one expression control sequence. Preferably, an expression control sequence is operably linked to a nucleic acid molecule such that the nucleic acid molecule can be expressed in an in vivo or in vitro transcription and/or translation system. The choice of expression control sequences is dependent upon the transcription system to be used. For example, if a prokaryotic organism such as E. coli is to be used to express a nucleic acid molecule, then at least one appropriate prokaryotic expression control sequence would be used. Likewise, if a eukaryotic organism is to be used to express a nucleic acid molecule, then at least one appropriate eukaryotic expression control sequence, such as CMV or LTRs would be used. Such nucleic acid molecules can be in any form, such as in a plasmid or in a linear form.

A nucleic acid molecule of the present invention can be provided with or without expression control sequences in a vector, such as a plasmid or a viral vector. Viral vectors can be chosen so that they are appropriate for a cell to be transfected, such as, for example, a phage, cosmid, retrovirus, vacinia, adenovirus or adenoassociated virus. Viral vectors can introduce a nucleic acid molecule into a cell during its normal biological proceses. Non-viral vectors can be used to introduce a nucleic acid molecule of the present invention into a host cell using methods known in the art, such as lipofection, cold calcium chloride or electroporation. The nucleic acid molecule in a cell can be extrachromosomal or be integrated into the genome of the cell. The host cell can be any appropriate host cell, such as a eukaryotic or prokaryotic cell.

As set forth in the Examples and exemplified in SEQ ID NO:1 and SEQ ID NO:3 or portions thereof or nucleic acid molecules including at least a portion thereof, nucleic acid molecules of the present invention can encode peptides that encode PKS activity. Nucleic acid molecules having PKS activity, or other activities associated with PKS, can be identified by making comparisons of nucleic acid sequence or translation amino acid sequences derived therefrom using methods known in the art, including BLAST comparisons. A nucleic acid molecule of the present invention can be expressed and the expression products screened and confirmed for having PKS activity. In addition, nucleic acid molecules of the present invention can encode polypeptides that have other activities of PKS. Methods for screening such activities are known in the art.

The nucleic acid molecules of the present invention can be used for a variety of applications, including but not limited to, PCR primers, probes to identify similar sequences, and to make polypeptides of the present invention. The particular application of a nucleic acid molecule depends on a variety of factors known in the art, such as the length, strandedness (single stranded or double stranded and positive sense or negative sense), chemical characterization (such as DNA or RNA) or whether the nucleic acid molecule is detectably labeled or not.

As set forth in the Examples and exemplified in SEQ ID NO: 2 and SEQ ID NO: 4 or portions thereof or polypeptides or proteins including at least a portion thereof, polypeptides encoding PKS activity have been isolated. The PK activity of polypeptides of the present invention can be screened and confirmed using methods known in the art.

The polypeptide of the present invention can be made using recognized methods, such as by way of recombinant methods as they are known in the art or by digesting proteins or polypeptides. For example, nucleic acid molecules encoding or suspected of encoding a polypeptide of the present invention can be cloned into expression vectors that are transformed into appropriate host cells where the nucleic acid molecules are expressed. The resulting polypeptides can be optionally purified and their activity confirmed using methods of the present invention or as they are known in the art or later developed. Alternatively, the in vivo activity of polypeptides can be confirmed using methods of the present invention or as they are known in the art.

A polypeptide of the present invention can be provided ex vivo or within a cell. A polypeptide of the present invention can be expressed within a cell by transfecting a cell with a nucleic acid molecule that encodes a polypeptide of the present invention. The nucleic acid molecules of the present invention can be operably linked to expression control sequences appropriate for the cell such that the nucleic acid molecule of the present invention is expressed on or within the cell. In this instance, a fusion protein that includes a detectable label as the polypeptide of interest can be used to track the location of the fusion protein in the cell.

Allelic variants of a polypeptide of the invention can readily be identified as having a high degree (significant) of sequence homology/identity to at least a portion of the polypeptide of the invention as well as being encoded by the same genetic locus as the polypeptide of the invention. Genetic locus can readily be determined based on the genomic information provided in, for example, the genomic sequence mapped to the B. neretina, E. sertula organism. As used herein, two proteins (or regions of the proteins) have significant homology when the amino acid sequences are typically at least about 70%-80%, 80%-90%, and more typically, at least about 90%-95% or more homologous. A significantly homologous amino acid sequence, according to the present invention, will be encoded by a nucleic acid sequence that will hybridize to a nucleic acid molecule encoding a polypeptide of the invention under stringent conditions as understood in the art.

The present invention further provides fragments of the polypeptide of the invention, in additiori to proteins and peptides that comprise and consist of such fragments, the fragments to which the invention pertains, however, are not to be construed as encompassing fragments that may be disclosed publicly prior to the present invention. As used herein, a fragment comprises at least 8, 10, 12, 14, 16, or more contiguous amino acid residues from a polypeptide of the invention. Such fragments can be chosen based on the ability to retain one or more of the biological activities of the polypeptide of the ivenntion or could be chosen for the ability to perform a function, e.g. bind a substrate or act as an immunogen. Particularly important fragments are biologically active fragments, peptides that are, for example, about 8 or more amino acids in length. Such fragments will typically comprise a domain or motif of the polypeptide of the invention, e.g. active site, a transmembrane domain or a substrate-binding domain. Further, possible fragments include, but are not limited to, domain or motif containing fragments, soluble peptide fragments, and fragments containing immunogenic structures. Predicted domains and functional sites are readily identifiable by computer programs well known and readily available to those of skill in the art. The present invention also includes a method of making a composition including providing at least one nucleic acid molecule or polypeptide of the present invention, and synthesizing at lest one polyketide or precursor thereof, such as a bryostatin.

At least one nucleic acid molecule of the present invention or at least one polypeptide of the present invention can be expressed and used in a system to synthesize a polyketide or precursor thereof, including a bryostatin. The polyketides or precursors thereof can be previously known or unknown polyketides or precursors thereof. A variety of methods of producing polyketides or precursors thereof using known PKS genes, in particular known PKS type I genes, have been reported. (See for Example U.S. Pat. No. 5,672,491; U.S. Pat. No. 5,712,146; U.S. Pat. No. 5,716,849; U.S. Pat. No. 5,744,350; U.S. Pat. No. 5,783,431; and U.S. Pat. No. 5,824,513).

Cells or extracts thereof (such as substantially purified extracts) that include one or more of the nucleic acid molecules of the present invention or one or more polypeptides of the present invention can be used to synthesize a wide variety of polyketides including bryostatins. Such cells or extracts thereof can be contacted with a variety of compounds, including substrates for PKS activity, particularly PKS activity present in the cells or extracts thereof. Polypeptides expressed from nucleic acids of the present invention can act on these compounds in order to make a wide variety of polyketides such as bryopyran rings including bryostatins. In one aspect of the present invention, more than one cell and/or extract thereof can be used in combination or sequentially such that the products made by combination of cells or extracts can be determined and its activity confirmed.

The present invention also includes compounds (bioactive or not bioactive) made or identified using the present invention. For example, the present invention includes polyketides and bryostatins made using at least one method of the present invention. A compound made or identified using a method of the present invention can be a novel or non-novel compound.

A compound of the present invention can be provided with at least one pharmaceutically acceptable carrier as they are known in the art and discussed herein. Such pharmaceutically acceptable carriers are known in the art and are disclosed herein. A compound of the present invention can also be a pharmaceutical composition.

The present invention also encompasses a bioactive compound or bioactivity in a pharmaceutical composition comprising a pharmaceutically acceptable carrier prepared for storage and preferably subsequent administration, which have a pharmaceutically effective amount of the bioactive compound or bioactivity in a pharmaceutically acceptable carrier or diluent. Acceptable carriers or diluents for therapeutic use are well known in the pharmaceutical art, and are described, for example, in Remington's Pharmaceutical Sciences, Mack Publishing Co., (A. R. Gennato edit. (1985)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The bioactive compounds and bioactivities of the present invention can be formulated and used as tablets, capsules or elixirs for oral administration; suppositories for rectal administration; sterile solutions, suspensions or injectable administration; and the like. Injectables can be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like. If desired, absorption enhancing preparation, such as lioposomes, can be used.

The pharmaceutically effective amount of a bioactive compound or bioactivity required as a dose will depend on the route of administration, the type of animal or patient being treated, and the physical characteristics of the specific animal under consideration. The dose can be tailored to achieve a desired effect, but will depend on such factors as weight, diet, concurrent medication and other factors which those skilled in the medical arts will recognize. In practicing the methods of the present invention, the pharmaceutical compositions can be used alone or in combination with one another, or in combination with other therapeutic or diagnostic agents. These products can be utilized in vivo, preferably in a mammalian patient, preferably in a human, or in vitro. In employing them in vivo, the pharmaceutical compositions can be administered to the patient in a variety of ways, including parenterally, intravenously, subcutaneously, intramuscularly, colonically, rectally, nasally or intraperiotoneally, employing a variety of dosage forms. Such methods can also be used in testing the activity of bioactive compounds or bioactivities in vivo.

As will be readily apparent to one skilled in the art, the useful in vivo dosage to be administered and the particular mode of administration will vary depending upon the age, weight and type of patient being treated, the particular pharmaceutical composition employed, and the specific use for which the pharmaceutical composition is employed. The determination of effective dosage levels, that is the dose levels necessary to achieve the desired result, can be accomplished by one skilled in the art using routine methods as discussed above. Typically, human clinical applications of products are commenced at lower dosage levels, with dosage level being increased until the desired effect is achieved. Alternatively, acceptable in vitro studies can be used to establish useful doses and routes of administration of the bioactive compounds and bioactivities.

It is believed that the novel KS genes of the invention shall provide a variety of utility in the art, including, for example, identification of the entire gene cluster responsible for bryostatin production. Such identification can be accomplished by, for example, BLASTING the genes against the symbiont genome database. In addition, the KS genes of the invention are also useful for assisting in the production of a Bryostatin by providing nucleic acid sequences that encode for polypeptide that catalyzes at least one step in the synthesis of a Bryostatin.

### EXAMPLES

### Example 1

### Identification of Biosynthetic PKS Gene

In this Example we examined the relationship between palatability, bryostatin concentration, and PKS gene expression in the reproductive and non-reproductive tissue of three adult colonies of B. neritina, and compare it to levels in larvae.

i) Collection of Adult and Larval B. neritina

Adult Bugula neritina were collected by SCUBA from Radio Island Jetty in Morehead City, N.C., USA, in Spring 2001 and 2002. Colonies were maintained in flowing seawater tables in the dark at UNC Chapel Hill's Institute of Marine Sciences. The adults were spawned by placing them in 4-L glass jars filled with seawater and exposing them to bright light in the morning (between 0800 and 1200). Adults released larvae from 30 min to 90 min after exposure to the light. Larvae swam to the top of the jar and were pipetted into chilled seawater.

ii) Adult and Larval Palatability and Bryostatin Concentration

The larvae were pooled and divided into three volumetric vials. They were spun for 5 min at 200 g (at 4° C.), and the excess seawater removed. The volume of larvae was measured and methanol was added to the vial until the larvae were extracted. Three adult colonies were separated into reproductive and non-reproductive branches, which differed only in the presence or absence of a calcareous brood chamber called the ovicell. The volume of each sample was measured, and the branches were cut into small pieces and placed into methanol until they were extracted. Adult and larval B. neritina were sequentially extracted once with methanol, four times with 1:1 mixture of dichloromethane and methanol, and then once more with methanol. These extracts were combined as the lipophilic extract, and evaporated to dryness. The lipophilic extracts were redissolved in the volume of solvent equivalent to the volume of tissue extracted for assays and HPLC analysis.

Palatability assays with the lipophilic extracts of larval and reproductive and non-reproductive adult tissues were performed with the omnivorous fish predator, Lagodon rhomboides, which are abundant in habitats where B. neritina live. Assays involved incorporating the extract into a volumetric amount of highly palatable squid paste with 2% sodium alginate. Control pellets are made with solvent instead of extract. For the assay, a control pellet was offered to an individual fish; next, an extract pellet was offered to the same fish, and the response of the fist (accept or reject) was recorded; finally, another control pellet was offered to the fish to ensure that the fish was not satiated and was still willing to feed. Only replicates with positive responses to both the control pellets were analyzed with McNemar's test for significance of changes. Differences between assays were tested with Fisher's exact test. The software SPSS 11.0 was used for statistical analyses.

The concentrations of deterrent bryostatins were determined via analytical HPLC with a C18 column (4.6×250 mm Microsorb 5 ¤m column, Varian, Walnut Creek, Calif.), elution with a gradient of methanol and water, and detection by a photodiode array (Waters Corp., Milford, Mass.). Standard curves were generated with known concentrations of the isolated bryostatins. Because bryostatin concentrations in the tissue were determined based on the amount of tissue extracted, and there were inherent differences in the morphological characteristics of adult and larval tissue, differences in bryostatin concentrations could be due to dilution of the tissue with structural material. To account for these differences, the mass of lipophilic and water soluble extracts, protein, mineral material, and chitin in dried tissue was measured, using three replicates of each tissue type. The percentage of the lipophilic extract compared to the percentage of proteins, mineral material, and chitin was calculated.

iii) Identification of the Polyketide Synthase Genes

DNA from larval B. neritina was extracted using Isoquick DNA extraction kit (Orca Research Inc., Bothell, Wash.). Degenerate primers for a PKS gene were developed based on Proteobacterial KS amino acid sequences deposited in GenBank [accession numbers: AF081920 (3 KS genes), AF239749 (2 KS genes), AF319998]. These were aligned using the ClustalW method, and regions of high homology were identified. The amino acid residues M/L/SDPQQR (SEQ ID NOs 5-7) were used for the forward primer, and LGDPIEI/L (SEQ ID NOs 8-9) for the reverse primer; the corresponding DNA sequences were aligned to determine the amount of degeneracy at each oligonucleotide position. Primers based on these sequences were used to amplify B. neritina larval DNA in a 25 μl PCR reaction with a “hot-start” Taq polymerase enzyme (JumpStart Taq, Sigma-Aldrich, St. Louis, Mo.). The first cycle consisted of a melting temperature at 94° C. for 2 min, annealing at 37° C. for 2 min, and extension at 72° C. for 3 min, followed by 39 cycles of denaturing for 30 sec, annealing at 54° C. for 45 sec, extension for 1 min, and a final extension for 5 min. The products were separated by electrophoresis on a 1% agarose gel, and a band at around 750 bp was excised from the gel. The PCR product was removed from the gel (Supelco GenElute Agarose Spin Column, Bellefont, Pa.) and cloned (Invitrogen TOPO TA Cloning Kit, Carlsbad, Calif.) into E. coli. There were seven resulting clones. They were screened by PCR using with the primers M13F and M13R, which amplify the insert. The clones that contained the insert were analyzed by restriction enzyme digestion with AluI. The five clones with unique inserts were sequenced using an Applied Biosystems 310 Genetic Analyzer (Foster City, Calif.). The resulting sequences were searched against the GenBank database. Internal primers based on the DNA sequences were synthesized and the clone DNA amplified with M13F and M13R. These products were sequenced and resulted in the complete DNA sequence of the insert. The full sequences were aligned with homologous sequences using ClustalX, and phylogenetic trees generated with Mega2.1 using the Minimum Evolution method and bootstrapping 10,000 times.

iv) Association of PKS Gene with E. Sertula

B. neritina larvae were treated with gentamicin for 10 days, transplanted to the field, and grown until they were reproductive adults. The next-generation larvae from experimental and control (larvae treated only with filtered seawater) adults were collected and extracted for DNA analysis. DNA was amplified first using E. sertula specific primers, and then primers developed from the PKS gene DNA sequence, BKSc1. The products were examined after electrophoresis through an agarose gel to assess the presence or absence of the gene.

v) Generation of CDNA and Quantitative Real-Time PCR

Tissue from the three B. neritina adults and two aliquots of larvae were preserved in RNAlater (Ambion Inc., Austin, Tex.) at −20° C. until RNA extraction. Tissue samples were rinsed in PBS twice, and then homogenized in 400 μl Solution D. RNA/DNA extraction followed the procedure. After homogenization, the samples were incubated at 65° C. for 5 min, and then spun at 12,500 rpm for 10 min. Three molar potassium acetate (pH 4.8) was added to the supernatant to make 0.14 M potassium acetate, and the mixture was incubated on ice for 30 min. After centrifuging for 10 min at 12.5 K rpm, 1/10 volume of 2 M sodium acetate, 1 volume of acid-phenol, and 0.5 volumes of 24:1 chloroform:isoamyl alcohol (IAA) were added and the samples were vortexed. After another spin at 12.5 K for 5 min, the aqueous layer was transferred to another tube, and re-extracted with phenol and chloroform:IAA. The aqueous layer was then extracted with 1 volume of chloroform. RNA was precipitated with 1 volume of isopropanol at −80° C. for >1 h, and then pelleted by centrifugation at 14 K rpm for 20 min. The pellet was rinsed with 75% ethanol and re-suspended in RNAase-free water.

Because the RNA extracted from adults was contaminated by pigments that inhibited enzymatic reactions, RNA from all of the tissues was subjected to another purification protocol (Ambion RNAqueous for PCR). After elution from the column, the RNA was treated with the enzyme DNase I to eliminate any DNA. The RNA was precipitated again, and quantified on a fluorometer (Shimadzu Scientific Instruments, Columbia, Md.) with SYBR Green II dye. Before cDNA was generated, the RNA was again treated with DNase I. Random hexamer primers and SuperScript II (Invitrogen, Carlsbad, Calif.) were used to make total cDNA from the RNA. To ensure that no DNA contaminated the reaction, a negative control was set up, with the only difference being no SuperScript was added. Both the positive and negative samples were amplified in a PCR reaction with primers specific to the cytochrome oxidase I (COI) gene of B. neritina, and the products separated by agarose gel electrophoresis.

The amount of PKS gene expression was determined using quantitative real-time PCR. In this method, the genes are amplified with a “hot-start” Taq polymerase (Brilliant SYBR Green QPCR Master Mix, Stratagene, LaJolla, Calif.); SYBR green I dye in the mix intercalates into the double stranded DNA, and the machine (Applied Biosystems Prism 7700 Sequence Detector) measures the fluorescence of the dye during the cycles. Lower concentration of target results in more PCR cycles before the dye fluoresces at the threshold intensity. The B. neritina COI and E. sertula 16S gene expression levels were used as proxies for bryozoan and symbiont cell numbers in each type of tissue, and the PKS gene expression was compared to each. The three genes were quantified in two larval cDNA. samples, and four adult samples (two reproductive and two non-reproductive). Standard curves were generated using a known concentration of PCR product for each set of primers. Samples and standards were run in triplicate. Because of the lower copy number of E. sertula genes (16S and PKS) compared to B. neritina genes, another standard curve using aposymbiotic B. neritina DNA spiked with the standards was generated. The two standard curves were compared, and any difference determined by regression analysis.

vi) Results

Palatability and Bryostatin 10 Concentration

The lipophilic larval extract significantly reduced pinfish, Lagodon rhomboides, feeding (P=0.001, N=13; McNemar's test). The feeding assays for the three replicate adult colonies were very similar: adult non-reproductive B. neritina extracts did not affect pinfish feeding (P>0.250), but adult reproductive extracts significantly reduced feeding by between 46 and 60% (P=0.031 and 0.016, FIG. 3.1). Only in adult 2 was there a significant difference between feeding responses to adult reproductive and non-reproductive extracts (P=0.018; Fisher's exact test). The feeding responses to larval, pooled adult reproductive, and pooled adult non-reproductive extracts were significantly different (P=0.003 between larval and adult reproductive, P<0.001 between larval and adult non-reproductive; Fisher's exact test). HPLC analysis revealed large differences in bryostatin concentrations in larval, adult reproductive and non-reproductive tissue. Because of the low levels of the bryostatins in adult tissues, the concentration of only bryostatin 10 could be measured with certainty.

Larvae have a significantly higher concentration of bryostatin 10 than both reproductive and non-reproductive adults (P=0.022 and 0.011, respectively; one-way ANOVA, Tukey's HSD; FIG. 3.2). Larval tissue has twice as much lipophilic extract as adult tissue (41.6%+2.3% SD and 20.4%+7.7% SD respectively, P=0.001; one-way ANOVA), while adults have almost twice as much protein, minerals and chitin than larvae (72.3%+1.0% SD and 41.3%+2.7% SD respectively, P=0.001; one-way ANOVA). However, this does not account for all of the difference in the bryostatin concentrations and palatability between adults and larvae.

PKS Genes and Expression Levels

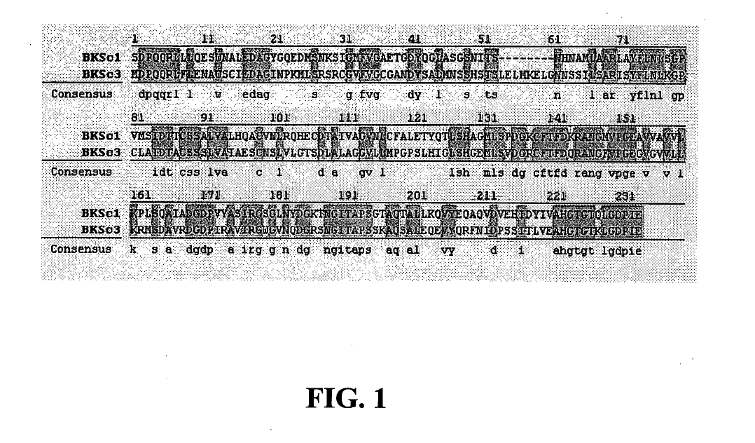

Of the 5 clones that had unique inserts of DNA, the sequences of two of the inserts had high homology to other bacterial polyketide syntheses; their amino acid sequences were 49.5% identical (FIG. 1). Interestingly, they were very similar to PKS genes from the Paederus beetle bacterial symbiont, a γ-Proteobacteria (Table 1). The two B. neritina/E. sertula clones grouped with other γ-Proteobacterial PKSs (FIG. 2).

Nucleotide sequences are as follows:

SEQ ID NO:1 (BKSc1)
gctcggacccccagcagcgcttgttgttacaggagtcatggaatgctctg
gaagatgccgggtatggacaagaagatatgagtaacaaaagtattggtat
gtttgtgggtgcggagacgggtgattatcagggtttagcaagtggcagta
atattacatccaatcataatgctatgcttgctgcacggctggcgtatttt
ctcaatctcagtggtccggtgatgtccattgatactacctgttcctccgc
tcttgttgctttacatcaagcctgtgtgaacttgcgtcagcacgaatgtg
atactgccatcgtagctggtgttaatttatgctttgcattggaaacctat
cagacattgagtcacgcaggtatgttatccccggatggcaaatgctttac
ctttgataagcgggccaatggtatggtccctggagaagcggtcgtcgcgg
tggtacttaaacccctctctcaggccattgcagacggtgatcccgtctat
gccagcattcgtggtagtggccttaactacgatggaaaaactaatggcat
taccgctcccagtggcacagcgcaaacagcgctgttgaaacaggtgtatg
aacaggcgcaagtggatgttgaacacattgactatattgtagcgcatggc
accggaacccaactgggcgacccaatcgaact
SEQ ID NO:3 (BKSc3)
Atggacccgcagcaacggttatttttagagaatgcgtg
gagttgtatagaggatgcggggattaaccctaagatgttatcccgtagtc
gatgtggggtatttgttgggtgcggtgcgaatgattacagcgctctaatg
aacagtagccactcaacgagtctcgaattaatgaaggaattaggcaacaa
ctcttccattttatctgcacgaatctcctactttttaaatttaaagggcc
cttgtcttgcgattgataccgcatgctcttcttcattagtggccattgcc
gagtcgtgtaatagtctggtgttgggtactagtgacttggcgttggcagg
tggagtgttgctgatgccaggtccatccttacatataggcttgagtcatg
gagaaatgttatcagtagatggtcgctgctttaccttcgatcaacgtgcc
aacggttttgtacctggagagggtgtaggggttgtcttgttaaaacgcat
gtcggatgcggtgcgtgatggtgatcccattcgtgcagtgatacggggct
ggggtgtgaatcaggatggtagaagtaatggtattacggcgccgagttca
aaggcgcaaagtgctctggagcaagaggtttatcaacgttttaatattga
tccatcgagcattaccttagtcgaagcacacggaaccggcaccaaactgg
gcgatcccatcgagct

Amino Acid Sequneces are as follows:

SEQ ID NO: 2 (BKSc1)
Ser Asp Pro Gln Gln Arg Leu Leu Leu Gln Glu Ser
trp Asn Ala Leu Glu Asp Ala Gly Tyr Gly Gln Glu
Asp Met Ser Asn Lys Ser Ile Gly Met Phe Val Gly
Ala Glu Thr Gly Asp Tyr Gln Gly Leu Ala Ser Gly
Ser Asn Ile Thr Ser Asn His Asn Ala Met Leu Ala
Ala Arg Leu Ala Tyr Phe Leu Asn Leu Ser Gly Pro
Val Met Ser Ile Asp Thr Thr Cys Ser Ser Ala Leu
Val Ala Leu His Gln Ala Cys Val Asn Leu Arg Gln
His Glu Cys Asp Thr Ala Ile Val Ala Gly Val Asn
Leu Cys Phe Ala Leu Glu Thr Tyr Gln Thr Leu Ser
His Ala Gly Met Leu Ser Pro Asp Gly Lys Cys Phe
Thr Phe Asp Lys Arg Ala Asn Gly Met Val Pro Gly
Glu Ala Val Val Ala Val Val Leu Lys Pro Leu Ser
Gln Ala Ile Ala Asp Gly Asp Pro Val Tyr Ala Ser
Ile Arg Gly Ser Gly Leu Asn Tyr Asp Gly Lys Thr
Asn Gly Ile Thr Ala Pro Ser Gly Thr Ala Gln Thr
Ala Leu Leu Lys Gln Val Tyr Glu Gln Ala Gln Val
Asp Val Glu His Ile Asp Tyr Ile Val Ala His Gly
Thr Gly Thr Gln Leu Gly Asp Pro Ile Glu
SEQ ID NO: 4 (BKSc3)
Met Asp Pro Gln Gln Arg Leu Phe Leu Glu Asn Ala
Trp Ser Cys Ile Glu Asp Ala Gly Ile Asn Pro Lys
Met Leu Ser Arg Ser Arg Cys Gly Val Phe Val Gly
Cys Gly Ala Asn Asp Tyr Ser Ala Leu Met Asn Ser
Ser His Ser Thr Ser Leu Glu Leu Met Lys Glu Leu
Gly Asn Asn Ser Ser Ile Leu Ser Ala Arg Ile Ser
Tyr Phe Leu Asn Leu Lys Gly Pro Cys Leu Ala Ile
Asp Thr Ala Cys Ser Ser Ser Leu Val Ala Ile Ala
Glu Ser Cys Asn Ser Leu Val Leu Gly Thr Ser Asp
Leu Ala Leu Ala Gly Gly Val Leu Leu Met Pro Gly
Pro Ser Leu His Ile Gly Leu Ser His Gly Glu Met
Leu Ser Val Asp Gly Arg Cys Phe Thr Phe Asp Gln
Arg Ala Asn Gly Phe Val Pro Gly Glu Gly Val Gly
Val Val Leu Leu Lys Arg Met Ser Asp Ala Val Arg
Asp Gly Asp Pro Ile Arg Ala Val Ile Arg Gly Trp
Gly Val Asn Gln Asp Gly Arg Ser Asn Gly Ile Thr
Ala Pro Ser Ser Lys Ala Gln Ser Ala Leu Glu Gln
Glu Val Tyr Gln Arg Phe Asn Ile Asp Pro Ser Ser
Ile Thr Leu Val Glu Ala His Gly Thr Gly Thr Lys
Leu Gly Asp Pro Ile Glu

TABLE 1
Homology of B. neritina/E. sertula KS gene amino
acid sequences with other bacterial PKS genes.

| | | | % | % | Accession |
|---|---|---|---|---|---|
| Clone | Gene | E-value | Identity | Similarity | No. |
| BKSc1 | Paederus symbiont PKS pedF | 3 × 10⁻⁸¹ | 67.0 | 78.4 | AAL27851 |
| | Polyangium cellulosum soraphen pksA | 2 × 10⁻⁵⁶ | 48.9 | 63.9 | AAK19883 |
| | Stigmatella aurantiaca myxalamid mxaF | 8 × 10⁻⁵⁶ | 49.4 | 65.7 | AF319998 |
| | Streptomyces atroolivaceus pks | ×10⁻⁵⁵ | 48.7 | 62.9 | AF484556 |
| | Microcystis aeruginosa microcystin mcyE | ×10⁻⁵³ | 47.6 | 65.2 | AF183408 |
| | Polyangium cellulosum epothilone epoF | ×10⁻⁵³ | 47.2 | 61.4 | AF217189 |
| | Stigmatella aurantiaca myxothiazol mtaB | ×10⁻⁵³ | 47.0 | 61.6 | AF188287 |
| | Planktothrix agardhii microcystin mcyE | ×10⁻⁵² | 47.6 | 62.2 | CAD29794 |
| BKSc3 | Pseudomonas fluorescens mupirocin MmpIV | 2 × 10⁻⁶² | 51.7 | 68.4 | AAM12913 |
| | Streptomyces atroolivaceus pks | ×10⁻⁶⁰ | 53.8 | 65.4 | AF484556 |
| | Polyangium cellulosum epothilone epoD | 8 × 10⁻⁶⁰ | 51.7 | 65.0 | AF217189 |
| | Polyangium cellulosum epothilone epoC | 8 × 10⁻⁶⁰ | 51.7 | 65.0 | AF210843 |
| | Paederus symbiont PKS pedF | 1 × 10⁻⁵⁹ | 48.9 | 69.8 | AAL27851 |
| | Stigmatella aurantiaca myxothiazol mtaB | ×10⁻⁵⁸ | 53.4 | 66.2 | AF188287 |
| | Stigmatella aurantiaca stigmatellin stiH | 6 × 10⁻⁵⁸ | 51.5 | 64.4 | CAD19092 |
| | Stigmatella aurantiaca myxalamid mxaE | 8 × 10⁻⁵⁸ | 51.7 | 67.5 | AF319998 |

FIG. 2 shows a minimum evolution tree of amino acid sequences of PKS genes from B. neritina/E. sertula and other related organisms (sequences obtained from GenBank). Bootstrap analysis (performed 10,000 times) percentages greater than 50% are labeled at the nodes. Scale bar indicates number of amino acid substitutions per site.

Only the BKSc1 gene was amplified with primers from cDNA generated with B. neritina larval RNA (FIG. 3). Therefore, only this PKS gene was chosen for quantitative expression analysis in adult and larval tissue. E. sertula loads were reduced by >99% in the aposymbiotic larvae, and the BKSc1 gene was not detected (FIG. 3). The standard curves generated by the pure PCR products and the products+B. neritina DNA were not significantly different. There were no significant differences in the expression of B. neritina COI gene in larval, adult reproductive, and adult non-reproductive tissue. The amounts of expression of E. sertula 16S rRNA gene per amount of expression of COI gene in both reproductive and non-reproductive adult tissue were higher than levels in larval tissue (FIG. 4). Along with the increase in 16S rRNA gene expression, there was an increase in BKSc1 gene expression per COI expression levels. However, there was no increase in the BKSc1 expression level per 16S rRNA expression (FIG. 4).

FIG. 3 shows the (a) Expression of PKS genes BKSc1 and BKSc3 (lanes 1-4 and lanes 5-8, respectively). Lanes 1 and 5=DNA control, lanes 2 and 6=larval cDNA RT+, lanes 3 and 7 =larval cDNA RT-, and lanes 4 and 8=no template control. (b) PCR of symbiotic and aposymbiotic B. neritina larval DNA with primers specific for the polyketide synthase gene, BKSc1. Lanes 1 and 2 are control (symbiotic) groups 1 and 2, and lanes 3, 4, and 5, are treatment (aposymbiotic) groups 1-3. Lane 6=no template control, and lane L=molecular weight ladder.

FIG. 4 sets forth a log of the ratios of B. neritina COI, E. sertula 16S and BKSc1 gene expression in B. neritina larval, adult reproductive and adult non-reproductive tissue. The standard deviations of the values are shown.

Although the invention is illustrated and described herein with reference to specific embodiments, the invention is not intended to be limited to the details shown. Rather, various modifications may be made in the details within the scope and range of equivalents of the claims and without departing from the invention

What is claimed:

1. An isolated nucleic acid molecule consisting of a nucleotide sequence selected from the group consisting of:
(a) a nucleotide sequence that encodes an amino acid sequence shown in SEQ ID NO:2;
(b) a nucleotide sequence that hybridizes under stringent conditions to a nucleic acid molecule shown in SEQ ID NO:1; and
(c) a nucleotide sequence that encodes a fragment of an amino acid sequence shown in SEQ ID NO:2, wherein said fragment comprises at least 10 contiguous amino acids.

2. The isolated nucleic acid molecule of claim 1 wherein said isolated nucleic acid encodes at least one polypeptide that catalyzes at least one step in the synthesis of a Bryostatin.

3. The isolated nucleic acid molecule of claim 2 wherein said polypeptide comprises a protein.

4. The isolated nucleic acid molecule of claim 1 wherein said isolated nucleic acid is obtained from a marine organism.

5. The isolated nucleic acid molecule of claim 4 wherein said isolated nucleic acid is obtained from a Bugula neretina organism.

6. The isolated nucleic acid molecule of claim 1 wherein said isolated nucleic acid is obtained from a bacterial symbiont.

7. The isolated nucleic acid molecule of claim 6 wherein said isolated nucleic acid is obtained from an Endobugula sertula symbiont.

8. The isolated nucleic acid molecule of claim 1 further comprising a nucleotide sequence that encodes an allelic variant of an amino acid sequence shown in SEQ ID NO:2.

9. A nucleic acid vector comprising the nucleic acid molecule of claim 1.

10. A host cell containing the vector of claim 9.

11. An isolated nucleic acid molecule consisting of a nucleotide sequence selected from the group consisting of:
(a) a nucleotide sequence that encodes an amino acid sequence shown in SEQ ID NO:4;
(b) a nucleotide sequence that hybridizes under stringent conditions to a nucleic acid molecule shown in SEQ ID NO:3; and
(c) a nucleotide sequence that encodes a fragment of an amino acid sequence shown in SEQ ID NO:4, wherein said fragment comprises at least 10 contiguous amino acids.

12. The isolated nucleic acid molecule of claim 11 wherein said isolated nucleic acid encodes at least one polypeptide that catalyzes at least one step in the synthesis of a Bryostatin.

13. The isolated nucleic acid molecule of claim 12 wherein said polypeptide comprises a protein.

14. The isolated nucleic acid molecule of claim 11 wherein said isolated nucleic acid is obtained from a marine organism.

15. The isolated nucleic acid molecule.of claim 14 wherein said isolated nucleic acid is obtained from a Bugula neretina organism.

16. The isolated nucleic acid molecule of claim 11 wherein said isolated nucleic acid is obtained from a bacterial symbiont.

17. The isolated nucleic acid molecule of claim 16 wherein said isolated nucleic acid is obtained from an Endobugula sertula symbiont.

18. The isolated nucleic acid molecule of claim 11 further comprising a nucleotide sequence that encodes an allelic variant of an amino acid sequence shown in SEQ ID NO:2.

19. A nucleic acid vector comprising the nucleic acid molecule of claim 11.

20. A host cell containing the vector of claim 19.
